(19) United States
(12) Patent Application Publication
(10) Pub. No.: US 2024/0011967 A1
(43) Pub. Date: Jan. 11, 2024

(54) SCREENING METHOD USING COATING COMPOSITION PROPERTIES OR WET FILM PROPERTIES

(71) Applicant: BASF COATINGS GMBH, Münster (DE)

(72) Inventors: Bernhard STEINMETZ, Münster (DE); Peggy JANKOWSKI, Würzburg (DE)

(21) Appl. No.: 18/247,439

(22) PCT Filed: Oct. 5, 2021
(86) PCT No.: PCT/EP2021/077414

§ 371 Date: Mar. 30, 2023

(30) Foreign Application Priority Data
Oct. 5, 2020 (EP) 20200110.3

(51) Int. Cl. G01N 33/32; B05D 7/00

(52) U.S. Cl. CPC G01N 33/32; B05D 7/534; B05D 2202/00; B05D 2503/00; B05D 2508/00

(57) ABSTRACT

Disclosed herein are a method of screening coating material compositions for development of new coating formulations in the automotive field, the method making use of measuring, selecting and improving at least one property of a coating material composition or of a wet coating film obtained therefrom, and a method for investigating the influence of certain constituents of coating material compositions on their properties or on the properties of resulting wet coating films obtained therefrom.

The present invention relates to a method of screening coating material compositions for development of new coating formulations in the automotive field, said method comprising at least steps (1), (4), (5), (8) and (10) to (13) and optionally at least one of steps (2), (3), (6), (7) and (9), as defined hereinafter, said method making use of measuring, selecting and improving at least one property of a coating material composition or of a wet coating film obtained therefrom, and to a use of said method for investigating the influence of certain constituents of coating material compositions on their properties or on the properties of resulting wet coating films obtained therefrom.

### BACKGROUND OF THE INVENTION

In typical automotive coating processes, usually at least four layers are applied to the surface of a suitable substrate such as a metallic substrate in form of a multilayer coating system: an electrodeposition coat (e-coat), at least one of a primer and a sealer, at least one basecoat, and a topcoat, in particular a clearcoat.

There are quite a number of requirements necessary, which have to be fulfilled and/or met by the coatings used in the automotive industry due to regulations, but also due to quality standards set by the automotive industry as such. Thus, the coatings obtained from using coating formulations in the automotive field have to exhibit or display a number of desired characteristics to at least a sufficient extent in order to meet these requirements. For example, an avoidance of optical defects and/or surface defects such as pinholes, mottling etc. is desired. In addition, the coatings are e.g. ought to have a good scratch resistance and a good stone chip resistance. In addition, it is often envisaged to lower the curing temperature as much as possible, in particular for economic reasons, but also in case temperature sensitive substrates are used such as plastic substrates. However, as outlined above in a conventional automotive multilayer coating system there are at least four kinds of different coating layers present and each layer is used for a different reason and to accomplish different desired characteristics of the overall coating. In addition, the coating formulations used for preparing each of the coating layers are usually rather complex, i.e., contain a comparably high number of different constituents. In addition, some of the coating formulations are predominantly provided in form of solventborne formulations such as clearcoats, whereas other coating formulations are provided to a high extent in the form of aqueous formulations such as basecoats. Thus, when constituents such as film-forming binders and crosslinking agents have been optimized for an incorporation into a solventborne system, these constituents are usually not optimized or even not applicable for a use in an aqueous system, and vice versa.

In practice, when developing new coating formulations, which are of rather complex nature as outlined above, in particular in research facilities of automotive manufacturers, it is therefore usually not only necessary to provide/produce the new complex coating formulations per se including testing if all constituents used or intended to be used have the desired compatibility, but it is usually also necessary to apply each of these complex coating formulations to a substrate, and to perform a testing or a series of testings with respect to the desired characteristics to be achieved based on these complex systems.

Thus, there is a need to provide a method of screening coating material compositions for development of new coating formulations in the automotive field in order to be able to provide coating films resulting therefrom with improved characteristics, which methods allows a development based on non-complex standard coating material compositions, but with a sufficient number of degrees of freedom in order to allow a screening of as many coating compositions as possible, in particular as far as the constituents such as film-forming polymers of the crosslinking agents present therein are concerned. At the same time said method should be able to be performed rather fast, cost-efficient and sustainable manner and in a manner saving resources (use of less amounts of materials and less waste production). In particular, there is a need to provide such a method, which allows an investigation of the influence of different constituents such as film-forming polymers and/or crosslinking agents used in such standard coating material compositions on the desired characteristics of the coating material compositions as such and also on wet coating films prepared therefrom in order to provide tailor made new coating formulations in a target-oriented manner, which in turn lead to coating films obtainable therefrom with improved properties. In this regard there is particularly a need to provide a method, which allows an investigation and an improvement of the properties of (wet) coating material compositions and wet coating films obtained therefrom based on the specific customer's needs and/or requirements—e.g. lowering the temperature needed for crosslinking to take place in order to save energy—, and which allows an adaptation of the (wet) coating material compositions and wet coating films employed according to the respective desired customer's coating processes.

### Problem

It has been therefore an object underlying the present invention to provide a method of screening coating material compositions for development of new coating formulations in the automotive field in order to be able to provide coating films resulting therefrom with improved characteristics, which methods allows a development based on non-complex standard coating material compositions, but with a sufficient number of degrees of freedom in order to allow a screening of as many coating compositions as possible in a defined time frame, in particular as far as the constituents such as film-forming polymers and/or crosslinking agents present therein are concerned. At the same time said method is ought to be able to be performed in a fast, cost-efficient and sustainable manner and in a manner saving resources (use of less amounts of materials and less waste production). In particular, it has been therefore an object underlying the present invention to provide such a method, which allows an investigation of the influence of different constituents such as film-forming polymers and/or crosslinking agents used in such standard coating material compositions on the desired characteristics of the coating material compositions as such and also on wet coating films obtainable therefrom in order to provide tailor made new coating formulations in a target-oriented manner, which in turn lead to coating films obtainable therefrom with improved properties. In this regard it has been in particular an object underlying the present invention to provide a method, which allows an investigation and improvement of the properties of (wet) coating material compositions and wet coating films obtained therefrom based on the specific customer's needs and/or requirements—e.g. lowering the temperature needed for crosslinking to take place in order to save energy—, and which allows an adaptation of the (wet) coating material compositions and wet coating films employed according to the respective desired customer's coating processes.

### Solution

This object has been solved by the subject-matter of the claims of the present application as well as by the preferred embodiments thereof disclosed in this specification, i.e. by the subject matter described herein.

A first subject-matter of the present invention is a method of screening coating material compositions for development of new coating formulations in the automotive field, said method comprising at least steps (1), (4), (5), (8) and (10) to (13) and optionally at least one of steps (2), (3), (6), (7) and (9), namely
(1) providing a first coating material composition F1,
   the first coating material composition F1 comprising
   (i) at least one film-forming polymer P1, said polymer P1 having crosslinkable functional groups,
   (ii) at least one crosslinking agent CA1 having functional groups, which are reactive at least towards the crosslinkable functional groups of polymer P1,
   (iii) water and/or at least one organic solvent,
   (iv) optionally at least one phase compatibilizing polymer PMP,
   (v) optionally at least one additive A, preferably at least one crosslinking catalyst CLC1 as additive A, which is capable of catalyzing the crosslinking reaction of the crosslinkable functional groups of polymer P1 with the functional groups of crosslinking catalyst CA1, and
   (vi) optionally at least one pigment P11 and/or filler F11, wherein the constituents (i), (ii), (iii), (iv) and (v) as well as (vi) are each different from one another,
(2) optionally applying the first coating material composition F1 onto at least one optionally pre-coated surface of a substrate to form a wet coating film on said surface of the substrate,
(3) optionally drying the wet coating film obtained after step (2) to form a partially dried coating film on the surface of the substrate,
(4) measuring at least one property of the coating material composition provided in step (1) and/or of the wet coating film if step (2) is performed and/or of the partially dried coating film if step (3) is performed,
(5) providing at least one further coating material composition being different from the first coating material composition F1 in precisely one or at most two, preferably precisely one, parameter(s),
   wherein said parameter(s) is/are selected from the group consisting of (a) partial or full exchange of polymer P1 with a further polymer being different from polymer P1 and having crosslinkable functional groups being reactive at least towards the functional groups of crosslinking agent CA1, (b) partial or full exchange of crosslinking agent CA1 with a further crosslinking agent being different from crosslinking agent CA1 and having functional groups being reactive at least towards the crosslinkable functional groups of polymer P1, (c) lowering or increasing the amount of polymer P1 and/or of the polymer being different from polymer P1, which has been used for partial or full exchange of polymer P1 as defined in (a) in the further coating material composition, (d) lowering or increasing the amount of crosslinking agent CA1 and/or of the crosslinking agent being different from CA1, which has been used for partial or full exchange of CA1 as defined in (b) in the further coating material composition, (e) partial or full exchange of additive A if present such as of a crosslinking catalyst CLC1 with a further additive being different from additive A such as a crosslinking catalyst being different from crosslinking catalyst CLC1, (f) lowering or increasing the amount of additive A if present such as of a crosslinking catalyst CLC1 and/or of the additive being different from additive A such as the of a crosslinking catalyst being different from CLC1, which has been used for partial or full exchange of additive A such as of crosslinking catalyst CLC1 as defined in (e) in the further coating material composition, (g) partial or full exchange of pigment P11 and/or filler F11 if present with a further pigment and/or filler being different from pigment P11 and/or filler F11, and (h) lowering or increasing the amount of pigment P11 and/or filler F11 if present and/or of the pigment and/or filler being different from pigment P11 and/or filler F11 if present, which has been used for partial or full exchange of pigment P11 and/or filler F11 as defined in (g) in the further coating material composition,
(6) optionally applying the further coating material composition onto at least one optionally pre-coated surface of a substrate to form a wet coating film on said surface of the substrate,
(7) optionally drying the wet coating film obtained after step (6) to form a partially dried coating film on the surface of the substrate,
(8) measuring at least one property of the coating material composition provided in step (5) and/or of the wet coating film if step (6) is performed and/or or of the partially dried coating film if step (7) is performed, said at least one property being the same property as measured in step (4),
(9) optionally repeating steps (5) and (8) and optionally step (6) and/or step (7) at least once, wherein each of the further coating compositions provided is different from one another and different from coating material composition F1,
(10) incorporating the results of the properties measured in steps (4) and (8) and optionally of the results of the properties measured in case of an at least one-fold repetition as defined in step (9) into a preferably electronic database,
(11) selecting at least one of the measured properties present in the preferably electronic database due to incorporation step (10),
(12) evaluating and comparing the results of the at least one selected property measured according to steps (4) and (8) and optionally after an at least one-fold repetition as defined in step (9) with each other, and
(13) using the information obtained from evaluation and comparison step (12) for formulating and providing at least one new coating formulation being different from the first coating material composition F1 and from each of the further coating material compositions obtained after step (5) and from any coating material composition obtained optionally after an at least one-fold repetition as defined in step (9), wherein the at least one new coating formulation as such and/or a wet or partially dried coating film obtained therefrom shows an improvement of the at least one property selected in step (11), when measured as defined in step (4) and/or (8).

Preferably, the parameter(s) selected in step (5) and, optionally, during an at least one-fold repetition as defined in step (9), is/are associated with partial or full exchange and/or lowering or increasing of the amount of at least one constituent that contributes to the total solid content of each of the coating material compositions, such as constituents (i) and (ii) and if present constituent (v) and/or (vi) in case of coating material composition F1, more preferably of at least one constituent that contributes to the total binder solid content of each of the coating material compositions, such as constituents (i) and (ii) and if present constituent (v) in case of coating material composition F1.

Preferably, none of steps (2), (3), (6) and (7) is performed. Thus, preferably, the at least one property measured in steps (4) and (8) and selected in step (11) is a property of a coating material composition as such and thus not a property of any wet or partially dried coating film obtained therefrom.

A further subject-matter of the present invention is a use of the inventive method for developing and providing new coating formulations in the automotive field, said new coating formulations preferably being basecoat compositions, in particular aqueous basecoat compositions.

A further subject-matter of the present invention is a use of the inventive method for investigating the influence of film-forming polymers (i), said polymers having crosslinkable functional groups, and/or of crosslinking agents (ii) having functional groups, which are reactive at least towards the crosslinkable functional groups of the polymers, and/or of additives (v) if present such as of crosslinking catalysts catalyzing a crosslinking reaction between (i) and (ii), and/or of pigments and/or fillers (vi) on properties of coating material compositions as such and/or of wet or partially dried coating films obtained therefrom, said coating material compositions containing at least constituents (i) and (ii) and optionally (v) and/or (vi). If crosslinking catalysts are used as additives (v) it is, of course, possible that these may exhibit further functions besides their catalytic activity. For example, in case blocked or unblocked sulfonic acids are used as crosslinking catalysts, these may also function as surfactants and thus also have an influence on the surface tension as a further example of a to be a measured property.

It has been surprisingly found that by the inventive method a screening of coating material compositions for development of new coating formulations in the automotive field with improved characteristics is available, which can be performed fast and in a cost-efficient manner. This is in particular the case as the method makes use of screening rather non-complex standard coating material compositions, preferably with only a limited number of constituents. Thus, the screening does not have to be performed by making use of rather complex coating formulations with a high number of different constituents, but can be based on said non-complex standard system, Therefore, the inventive method cannot only be practiced in a cost-efficient manner as only a limited number of constituents has to be used, but additionally resources can be saved as less amounts of constituent materials have to be used and less waste is produced. In addition, the inventive method has, therefore, advantages from an ecological point of view.

It has been further surprisingly found that the inventive method can be performed in a cost-efficient and sustainable manner as all results of the properties measured are incorporated into a preferably electronic database, which can be accessed at any time and which can be updated during the process of performing the inventive method. The more data are present in the database, the more efficient, promising and exact can steps (12) and (13) be performed.

It has been further surprisingly found that the inventive method allows a screening of a higher number of coating compositions in the same time interval as conventional screening methods.

In particular, it has been surprisingly found that the inventive method allows an investigation of the influence of constituents such as film-forming polymers and/or crosslinking agents present in the standard coating material compositions used on the desired characteristics of the coating material compositions as such and also on wet coating or partially dried films obtained therefrom and that in this manner tailor made new coating formulations can be provided in a target-oriented manner, which in turn have improved properties. This in particular applies to the investigation of hydroxyl- (OH—) and optionally also acid-functional polymers as film-forming polymers and/or melamine aldehyde resins as crosslinking agents and/or crosslinking catalysts catalyzing a crosslinking reaction between these two constituents as well as and their influence on desired characteristics such as the curing temperature and/or curing time needed for curing, which is in each case desired to be as low as possible. It has been found in this context that new tailor made coating formulations containing OH— and optionally also acid-functional polymers as film-forming polymers and melamine aldehyde resins as crosslinking agents and optionally at least one crosslinking catalyst can be provided in a target-oriented manner by the inventive method, which lead to new coating formulations having as such an improved property and/or wet or partially dried coating films obtained therefrom which have an improved property such as an improvement with respect to onset temperature and/or offset time that in turn leads to an improvement of at least one characteristic of the coating formulations and/or wet or partially dried coating films obtained therefrom such as higher reactivity and/or shorter reaction time and thus shorter curing time and/or lower curing temperature required for an appropriate processing of the coating formulation.

Moreover, it has been surprisingly found that the inventive method allows an investigation and improvement of the properties of (wet) coating material compositions and wet coating films obtained therefrom based on the specific customer's needs and/or requirements—e.g. lowering the temperature needed for crosslinking to take place in order to save energy—, and further allows an adaptation of the (wet) coating material compositions and wet coating films employed according to the respective desired customer's coating processes.

### DETAILED DESCRIPTION OF THE INVENTION

### Inventive Method

### Step (1)

In step (1) of the inventive method a first coating material composition F1 is provided. The first coating material composition F1 comprises at least constituents (i), (ii) and (iii) and optionally (iv) and/or (v) and/or (v). The constituents (i), (ii), (iii), (iv) and (v) as well as (vi) are different from one another.

The term “comprising” in the sense of the present invention, in connection for example with any of the inventively used coating material compositions such as F1 or any of the new coating formulations preferably has the meaning of “consisting of”. In this case it is possible—in addition to all mandatory constituents present therein—for one or more of the further constituents identified hereinafter to be also optionally included therein. All constituents may in each case be present in their preferred embodiments as identified hereinafter.

The proportions and amounts in wt.-% (% by weight) of any of the constituents given hereinafter, which are present in any of the coating material compositions add up to 100 wt.-%, based in each case on the total weight of each composition.

### Coating Material Composition F1 and Further Coating Material Compositions

Preferably, constituents (i) to (iii) and optionally (iv) and/or (v) and/or (vi) are the only constituents of the first coating material composition F1. Thus, preferably, the first coating material composition F1 consists of constituents (i) to (iii) and optionally (iv) and/or (v) and/or (vi). Preferably, constituent (iv) is present in the first coating material composition F1. The same preferably applies to each of the further coating material compositions different from F1.

Preferably, the first coating material composition F1 and any of the further coating material compositions provided in step (5) and, optionally, obtained after an at least one-fold repetition as defined in step (9), are one-component (1 K) or two-component (2K) coating material compositions, preferably are each one-component (1 K) coating material compositions, more preferably for use as basecoat material compositions. The basecoat material compositions can be aqueous (waterborne) or organic solvent-based (solventborne, non-aqueous) and can be used both as OEM coating material compositions and for refinish applications. The same preferably applies to each of the further coating material compositions different from F1.

The term “basecoat” is known in the art and, for example, defined in Römpp Lexikon, paints and printing inks, Georg Thieme Verlag, 1998, 10th edition, page 57. A basecoat is therefore in particular used in automotive painting and general industrial paint coloring in order to give a coloring and/or an optical effect by using the basecoat as an intermediate coating composition. This is generally applied to a metal or plastic substrate, optionally pretreated with a primer and/or filler and/or sealer, sometimes in the case of plastic substrates also directly on the plastic substrate, and in the case of metal substrates on an electrodeposition coating layer coated onto the metal substrate, wherein the metal substrate may already bear a phosphate layer such as a zinc phosphate layer, or on the metal substrate already bearing a primer and/or filler and/or sealer and/or electrodeposition coating, or to already existing coatings in case of refinish applications, which can also serve as substrates. In order to protect a basecoat film in particular against environmental influences, at least one additional topcoat film, on particular clearcoat film is applied to it.

Preferably, the coating material composition F1 has a total solid content (non-volatile content) in the range of from 10 to 85 wt.-%, more preferably of from 15 to 80 wt.-%, even more preferably of from 20 to 65 wt.-%. The same preferably applies to each of the further coating material compositions different from F1. The total solid content is determined according to the method described in the ‘Methods’ section. Preferably, the total solid content in each case exceeds 40 wt.-%, more preferably exceeds 50 wt.-%.

### Film-Forming Polymers and Crosslinking Agents

The first coating material composition F1 comprises at least one film-forming polymer P1, said polymer P1 having crosslinkable functional groups, as constituent (i). The first coating material composition F1 further comprises at least one crosslinking agent CA1 having functional groups, which are reactive at least towards the crosslinkable functional groups of polymer P1, as constituent (ii). A crosslinking reaction taking preferably place between the crosslinkable functional groups of polymer P1 and the functional groups of crosslinking agent CA1 can but does not necessarily have to be catalyzed by at least one crosslinking catalyst (optional constituent (v). If such a catalysis is desired the first coating material composition F1 additionally comprises at least one crosslinking catalyst (v). The same preferably applies to each of the further coating material compositions different from F1.

### Film-Forming Polymers

The film-forming polymer P1 represents a binder. For the purposes of the present invention, the term “binder” is understood in accordance with DIN EN ISO 4618 (German version, date: March 2007) to be the non-volatile (solid) constituent of a coating material composition, which is responsible for the film formation. The term includes crosslinking agents (crosslinkers) and additives such as catalysts if these represent non-volatile constituents. Pigments and/or fillers are not subsumed under the term “binder” as these are not responsible for film formation. Preferably, however, no pigments and/or fillers are present. Preferably, each of constituents (i), (ii), (iv) and (v) contributes to the total binder solids content of the coating material composition F1. The same preferably applies to each of the further coating material compositions different from F1.

Preferably, the at least one polymer P1 is the main binder of the coating material composition. As the main binder in the sense of the present invention, a binder constituent is preferably referred to, when there is no other binder constituent in the coating material composition, which is present in a higher proportion based on the total weight of the coating material composition.

The term “polymer” is known to the person skilled in the art and, for the purposes of the present invention, encompasses polyadducts and polymerizates as well as polycondensates. The term “polymer” includes both homopolymers and copolymers.

Suitable polymers which can be used as film-forming polymers are, for example, known from EP 0 228 003 A1, DE 44 38 504 A1, EP 0 593 454 B1, DE 199 48 004 A1, EP 0 787 159 B1, DE 40 09 858 A1, WO 92/15405 A1, WO 2005/021168 A1, WO 2017/097642 A1 and WO 2017/121683 A1.

Preferably, the at least one polymer P1 present as constituent (i) in the first coating material composition F1 and any further at least one film-forming polymer being present in any of the further coating material compositions provided in step (5) and, optionally, obtained after an at least one-fold repetition as defined in step (9), is selected from the group consisting of physically drying polymers, chemically crosslinking polymers, radiation curing polymers, and mixtures thereof, more preferably is selected from the group consisting of physically drying polymers, chemically self-crosslinking polymers, chemically non-self-crosslinking polymers (i.e. externally crosslinking polymers), radiation curing polymers, and mixtures thereof, even more preferably is selected from the group consisting of chemically non-self-crosslinking polymers.

Radiation curing polymers are curable via inducing radiation. Preferably, such polymers contain functional groups comprising carbon-carbon double bonds, e.g. vinyl and/or (meth)acryl groups. Physically drying polymers are primarily curable, preferably are curable, via physical drying. The presence of crosslinkable functional groups such as acid groups within these polymers is not in contradiction to the fact that they are primarily cured or cured by physical drying. Even in case of a (primarily) physical drying these polymers can additionally undergo at least partially a crosslinking reaction with the at least one crosslinking agent. Chemically crosslinking polymers are in particular chemically self-crosslinking polymers and chemically non-self-crosslinking polymers (i.e. externally crosslinking polymers). Even in case of (primarily) a self-crosslinking reaction, these polymers can additionally at least partially undergo a crosslinking reaction with the at least one crosslinking agent. Particularly preferred are, however, chemically non-self-crosslinking polymers.

The polymer P1 has crosslinkable functional groups, which enable a crosslinking reaction with the functional groups of crosslinking agent CA1. Any common suitable crosslinkable reactive functional group known to those skilled in the art can be present. Preferably, the polymer P1 has at least one kind of functional reactive groups selected from the group consisting of hydroxyl groups (also referred to herein as OH-groups), amino groups such as primary and/or secondary amino groups, thiol groups, epoxide groups, keto groups including diketo groups, acid groups such as carboxyl groups, and carbamate groups, siloxane groups, as well as functional groups comprising carbon-carbon double bonds, e.g. vinyl and/or (meth)acryl groups. Preferably, the polymer P1 has functional hydroxyl groups and/or acid groups, in particular hydroxyl and/or carboxylic acid groups, most preferably hydroxyl groups. Preferably, the same applies to any polymers used for partial or full exchange of polymer P1 as defined in step (5) of the inventive method, and similarly in case of an at least one-fold repetition as defined in optional step (9).

Preferably, the polymer P1 is hydroxyl-functional and more preferably has an OH number in the range of 5 to 400 mg KOH/g, more preferably from 7.5 to 350 mg KOH/g, more preferably of from 10 to 300 mg KOH/g. Preferably, the polymer P1 is additionally or alternatively acid-functional and more preferably has an acid number in the range of 0 to 200 mg KOH/g, more preferably from 1 to 150 mg KOH/g, even more preferably of from 1 to 100 mg KOH/g. Preferably, the same applies to any polymers used for partial or full exchange of polymer P1 as defined in step (5) of the inventive method, and similarly in case of an at least one-fold repetition as defined in optional step (9).

Preferably, the at least one polymer P1 present as constituent (i) in the first coating material composition F1 and any further at least one polymer being present in any of the further coating material compositions provided in step (5) and, optionally, obtained after an at least one-fold repetition as defined in step (9), are selected from the groups consisting of polyesters, poly(meth)acrylates, polyurethanes, polyureas, polyamides and polyethers, preferably are selected from the groups consisting of polyesters, poly(meth)acrylates, polyurethanes and polyethers. This includes copolymers containing structural units of said homopolymers such as polyurethane-poly(meth)acrylates. The same preferably applies to each of the film-forming polymers present in any of the further coating material compositions different from F1.

Most preferred are polyesters, poly(meth)acrylates, polyurethanes and polyethers, each having at least OH-groups as crosslinkable functional groups, and optionally additionally having acid groups.

The term “(meth) acryl” or “(meth) acrylate” or (meth)acrylic” in the context of the present invention in each case comprises the meanings “methacryl” and/or “acryl” “methacrylic” and/or “acrylic” or “methacrylate” and/or “acrylate”. Therefore, a “(meth)acrylic copolymer” in general may be formed from only “acrylic monomers”, only “methacrylic monomers” or “acrylic and methacrylic monomers”. However, polymerizable monomers other than acrylic and/or methacrylic monomers as e.g. styrene and the like may also be contained in a “(meth)acrylic copolymer”. In other words, a (meth)acrylic polymer may consist of only acrylic and/or methacrylic monomer units but does not have to. The notation “(meth)acrylate polymer or copolymer” or “(meth)acrylic polymer or copolymer” is intended to mean that the polymer/copolymer (polymer skeleton/backbone) is formed predominantly, i.e. preferably more than 50 mol-% or more than 75 mol-% of the monomer units used, from monomers having a (meth)acrylate group. In the preparation of a (meth)acrylic copolymer, preferably more than 50 mol-% or 75 mol-% of the monomers thus have a (meth)acrylate group. However, the use of further monomers as comonomers such as copolymerizable vinyl monomers, e.g. styrene, for its preparation is not excluded.

Preferred polyurethanes are described, for example, in German patent application DE 199 48 004 A1, page 4, line 19 to page 11, line 29 (polyurethane prepolymer B1), in European patent application EP 0 228 003 A1, page 3, line 24 to page 5, Line 40, European Patent Application EP 0 634 431 A1, page 3, line 38 to page 8, line 9, and international patent application WO 92/15405, page 2, line 35 to page 10, line 32.

Preferred polyesters are described, for example, in DE 4009858 A1 in column 6, line 53 to column 7, line 61 and column 10, line 24 to column 13, line 3 or WO 2014/033135 A2, page 2, line 24 to page 7, line 10 and page 28, line 13 to page 29, line 13 described. Likewise, preferred polyesters are polyesters having a dendritic structure or star-shaped structure, as described, for example, in WO 2008/148555 A1.

Preferred polyethers are, e.g., described in WO 2017/097642 A1 and WO 2017/121683 A1.

Preferred polyurethane-poly(meth)acrylate copolymers (e.g., (meth)acrylated polyurethanes)) and their preparation are described, for example, in WO 91/15528 A1, page 3, line 21 to page 20, line 33.

Particularly preferred are (meth)acrylic copolymers, in particular when they are OH-functional. Hydroxyl-containing monomers include hydroxy alkyl esters of acrylic or methacrylic acid, which can be used for preparing the copolymer. Non-limiting examples of hydroxyl-functional monomers include hydroxyethyl(meth)acrylate, hydroxypropyl(meth)acrylates, hydroxybutyl-(meth)acrylates, hydroxyhexyl(meth)-acrylates, propylene glycol mono(meth)acrylate, 2,3-dihydroxypropyl(meth)acrylate, pentaerythritol mono(meth)acrylate, polypropylene glycol mono(meth)acrylates, polyethylene glycol mono(meth)acrylates, reaction products of these with epsilon-caprolactone, and other hydroxyalkyl-(meth)acrylates having branched or linear alkyl groups of up to about 10 carbons, and mixtures of these. Hydroxyl groups on a vinyl polymer such as an (meth)acrylic polymer can be generated by other means, such as, for example, the ring opening of a glycidyl group, for example from copolymerized glycidyl methacrylate, by an organic acid or an amine. Hydroxyl functionality may also be introduced through thio-alcohol compounds, including, without limitation, 3-mercapto-1-propanol, 3-mercapto-2-butanol, 11-mercapto-1-undecanol, 1-mercapto-2-propanol, 2-mercaptoethanol, 6-mercapto-1-hexanol, 2-mercaptobenzyl alcohol, 3-mercapto-1,2-proanediol, 4-mercapto-1-butanol, and combinations of these. Any of these methods may be used to prepare a useful hydroxyl-functional (meth)acrylic polymer. Examples of suitable comonomers that may be used include, without limitation, α,β-ethylenically unsaturated monocarboxylic acids containing 3 to 5 carbon atoms such as acrylic, methacrylic, and crotonic acids and the alkyl and cycloalkyl esters, nitriles, and amides of acrylic acid, methacrylic acid, and crotonic acid; α,β-ethylenically unsaturated dicarboxylic acids containing 4 to 6 carbon atoms and the anhydrides, monoesters, and diesters of those acids; vinyl esters, vinyl ethers, vinyl ketones, and aromatic or heterocyclic aliphatic vinyl compounds. Representative examples of suitable esters of acrylic, methacrylic, and crotonic acids include, without limitation, those esters from reaction with saturated aliphatic alcohols containing 1 to 20 carbon atoms, such as methyl, ethyl, propyl, isopropyl, n-butyl, isobutyl, tert-butyl, hexyl, 2-ethylhexyl, dodecyl, 3,3,5-trimethylhexyl, stearyl, lauryl, cyclohexyl, alkyl-substituted cyclohexyl, alkanol-substituted cyclohexyl, such as 2-tert-butyl and 4-tert-butyl cyclohexyl, 4-cyclohexyl-1-butyl, 2-tert-butyl cyclohexyl, 4-tert-butyl cyclohexyl, 3,3,5,5,-tetramethyl cyclohexyl, tetrahydrofurfuryl, and isobornyl acrylates, methacrylates, and crotonates; unsaturated dialkanoic acids and anhydrides such as fumaric, maleic, itaconic acids and anhydrides and their mono- and diesters with alcohols such as methanol, ethanol, propanol, isopropanol, butanol, isobutanol, and tert-butanol, like maleic anhydride, maleic acid dimethyl ester and maleic acid monohexyl ester; vinyl acetate, vinyl propionate, vinyl ethyl ether, and vinyl ethyl ketone; styrene, a-methyl styrene, vinyl toluene, 2-vinyl pyrrolidone, and p-tert-butylstyrene. The (meth)acrylic copolymer may be prepared using conventional techniques, such as by heating the monomers in the presence of a polymerization initiating agent and optionally a chain transfer agent.

Suitable poly(meth) acrylates are also those which can be prepared by multistage free-radical emulsion polymerization of olefinically unsaturated monomers in water and/or organic solvents. Examples of seed-core-shell polymers (SCS polymers) obtained in this manner are disclosed in WO 2016/116299 A1.

Preferably, the at least one polymer P1 is present in the coating material composition F1 in an amount in a range of from 10.0 to 90 wt.-%, more preferably of from 15.0 to 85 wt.-%, even more preferably of from 17.5 to 65 wt.-%, still more preferably of from 20.0 to 60 wt.-%, in each case based on the total binder solids content of the coating material composition. Preferably, the same applies to any polymers used for partial or full exchange of polymer P1 as defined in step (5) of the inventive method, and similarly in case of an at least one-fold repetition as defined in optional step (9), with respect to any of the further coating material compositions different from F1 and from one another.

Preferably, the at least one polymer P1 is present in the coating material composition F1 in an amount in a range of from 50.0 to 90 wt.-%, more preferably of from 55.0 to 85 wt.-%, even more preferably of from 57.5 to 80 wt.-%, in each case based on the total solids content of the coating material composition. Preferably, the same applies to any polymers used for partial or full exchange of polymer P1 as defined in step (5) of the inventive method, and similarly in case of an at least one-fold repetition as defined in optional step (9), with respect to any of the further coating material compositions different from F1 and from one another.

### Crosslinking Agents

All conventional crosslinking agents can be used. This includes melamine resins, preferably melamine aldehyde resins, more preferably melamine formaldehyde resins, blocked polyisocyanates, polyisocyanates having free (unblocked) isocyanate groups, crosslinking agents having amino groups such as secondary and/or primary amino groups, and crosslinking agents having epoxide groups and/or hydrazide groups, as well as crosslinking agents having carbodiimide groups, as long as the functional groups of the particular crosslinking agent are suitable to be reacted with the crosslinkable functional groups of the film-forming polymers used as binders in a crosslinking reaction. For example, a crosslinking agent having blocked or free isocyanate groups can be reacted with a film-forming polymer having crosslinkable OH-groups and/or amino groups at elevated temperatures in case of 1K formulations and at ambient temperature in case of 2K formulations. Further possible combinations are epoxide groups, for example of a film-forming polymer, which can be reacted with acid and/or amino groups of a crosslinking agent, or vice versa. Further possible combinations are keto groups, for example of a film-forming polymer, which can be reacted with hydrazide groups of a crosslinking agent, or vice versa, or carbodiimde groups, for example of a crosslinking agent, which can be reacted with acid groups of a film-forming polymer or vice versa.

Preferably, the at least one crosslinking agent CA1 present as constituent (ii) in the first coating material composition F1 and any further at least one crosslinking agent being present in any of the further coating material compositions provided in step (5) and, optionally, obtained after an at least one-fold repetition as defined in step (9), are melamine aldehyde resins, preferably melamine formaldehyde resins. This in particular applies in case of 1K coating material compositions. As outlined hereinbefore crosslinking agents are to be included among the film-forming non-volatile components of a material coating composition, and therefore fall within the general definition of the “binder” as mentioned hereinbefore.

Preferably, the melamine aldehyde resins, preferably the melamine formaldehyde resins, in each case bear at least one of imino groups, alkykol groups and etherified alkylol groups as functional groups, which are reactive towards the functional groups of polymer P1. Examples of alkylol groups are methylol groups.

At least some of the alkylol groups present in the melamine aldehyde resins may be alkylated through further reaction with at least one alcohol to produce nitrogen-bonded alkoxyalkyl groups (etherified alkylol groups). In particular, the hydroxyl groups in the nitrogen-bonded alkylol groups may be reacted with the alcohol through an etherification reaction to produce nitrogen-bonded alkoxyalkyl groups. The alkoxyalkyl groups are available for a crosslinking reaction with, for example, suitable crosslinkable functional groups of polymer P1 such as OH− and/or acid groups. The remaining imino groups present after the aldehyde/melamine reaction are unreactive with the alcohol used for alkylation.

As outlined above the alkylol groups of the melamine aldehyde resins may be partially alkylated. By “partially alkylated”, it is meant that a sufficiently low amount of alcohol is reacted with the melamine aldehyde resins to leave some of the alkylol groups in the melamine aldehyde resins, under reaction conditions that should result in incomplete alkylation of the alkylol groups. When the melamine aldehyde resins are partially alkylated, they are typically alkylated with alcohol in amounts sufficient to leave alkylol groups present in the aminoplast in an amount of at least about 2%, more preferably of from about 10% to about 50%, even more preferably of from about 15% to about 40%, in each case based on the total number of reactive sites present in the melamine prior to reaction. Typically, the melamine aldehyde resin is partially alkylated to obtain from about 40 to about 98% of alkoxyalkyl groups, more preferably of from about 50% to about 90%, even more preferably of from about 60% to about 75%, in each case based on the total number of reactive sites present in the melamine prior to reaction.

Preferably, at least a portion, more preferably only a portion, of the alkylol groups such as methylol groups of the melamine aldehyde resin is etherified by reaction with at least one alcohol. Any monohydric alcohol can be employed for this purpose, including methanol, ethanol, n-propanol, iso-propanol, n-butanol, sec-butanol, t-butanol, pentanol, hexanol, heptanol, as well as benzyl alcohol and other aromatic alcohols, cyclic alcohols such as cyclohexanol, monoethers of glycols, and halogen-substituted or other substituted alcohols such as 3-chloropropanol and butoxyethanol. In particular, at least a part of the alkylol groups of the melamine aldehyde resin is partially modified with methanol and/or n-butanol and/or iso-butanol.

Preferably, constituent (ii) is present in the first coating material composition F1 in an amount in a range of from 7.5 to 45 wt.-%, more preferably of from 10.0 to 40 wt.-%, even more preferably of from 12.5 to 35 wt.-%, in particular of from 15 to 40 wt.-%, in each case based on the total binder solids of the coating material composition. Preferably, the same applies to any crosslinking agents used for partial or full exchange of crosslinking agent CA1 as defined in step (5) of the inventive method, and similarly in case of an at least one-fold repetition as defined in optional step (9), with respect to any of the further coating material compositions different from F1 and from one another.

Preferably, the at least one crosslinking agent CA1 is present in the coating material composition F1 in an amount in a range of from 10.0 to 50 wt.-%, more preferably of from 15.0 to 45 wt.-%, even more preferably of from 20.5 to 42.5 wt.-%, in each case based on the total solids content of the coating material composition. Preferably, the same applies to any crosslinking agents used for partial or full exchange of crosslinking agent CA1 as defined in step (5) of the inventive method, and similarly in case of an at least one-fold repetition as defined in optional step (9), with respect to any of the further coating material compositions different from F1 and from one another.

Preferably, the at least one film-forming polymer P1 and the at least one crosslinking agent CA1 are present in the coating material composition in a relative weight ratio, based on their solids, in a range of from 9.0:1 to 1.2:1, in particular of from 8.5:1.5 to 1.5:1. Preferably, the same applies to any crosslinking agents used for partial or full exchange of crosslinking agent CA1 as defined in step (5) of the inventive method, and similarly in case of an at least one-fold repetition as defined in optional step (9), with respect to any of the further coating material compositions different from F1 and from one another.

Constituent (iii)

The first coating material composition F1 comprises water and/or at least one organic solvent as constituent (iii).

All conventional organic solvents known to those skilled in the art can be used as organic solvents. The term “organic solvent” is known to those skilled in the art, in particular from Council Directive 1999/13/EC of 11 Mar. 1999. Examples of such organic solvents would include heterocyclic, aliphatic, or aromatic hydrocarbons, mono- or polyhydric alcohols, especially methanol and/or ethanol, ethers, esters, ketones, and amides, such as, for example, N-methylpyrrolidone, N-ethylpyrrolidone, dimethylformamide, toluene, xylene, butanol, ethyl glycol and butyl glycol and also their acetates, butyl diglycol, diethylene glycol dimethyl ether, cyclohexanone, methyl ethyl ketone, methyl isobutyl ketone, acetone, isophorone, or mixtures thereof. Preferably, however, no mono- or polyhydric alcohols are used.

Depending on the content and amount of water and/or organic solvent(s) the coating material composition can be “solventborne” (“non-aqueous”) or “waterborne” (“aqueous”). The term “solventborne” or “non-aqueous” is understood preferably for the purposes of the present invention to mean that organic solvent(s), used as solvent and/or as diluent, is/are present as the main constituent of all solvents and/or diluents present in the coating material composition. If the coating material composition is solventborne, constituent (iii) preferably is at least one organic solvent and does not represent water or comprises an amount of water being less than the amount of organic solvent(s). The coating composition when solventborne preferably is free or essentially free of water. The term “waterborne” or “aqueous” is understood preferably for the purposes of the present invention to mean that water is present as the main constituent of all solvents and/or diluents present in the inventive coating material composition. If it is waterborne, constituent (iii) comprises water at least as main solvent/diluent. Organic solvent(s) may be additionally present, but in a lower amount than water.

Preferably, the same applies to any of the further coating material compositions different from F1 and from one another provided according to step (5) of the inventive method, and similarly in case of an at least one-fold repetition as defined in optional step (9).

### Constituent (iv)

The first coating material composition F1 optionally and preferably comprises at least one phase compatibilizing polymer PMP as constituent (iv). Phase compatibilizing polymer PMP is used as phase mediator and/or phase promoter in particular in case both water and at least one organic solvent are present in the first coating material composition. Preferably, constituent (iv) is present in the first coating material composition F1, when constituent (iii) represents at least partially water.

Constituent (iv) preferably exhibits phase compatabilizing properties, i.e. has tenside characteristics. Constituent (iv) is, however, only an optional constituent as the desired compatabilizing properties may alternatively be introduced into the coating material composition via constituent (i). Thus, polymer P1 may not only function as film-forming polymer, but additionally also as phase mediator/phase promoter, in which the presence of optional constituent (iv) is not necessary. Furthermore, the presence of constituent (iv) may in particular be not necessary when the crosslinking agent present such as CA1 is a water-dilutable crosslinking agent such as a water-dilutable melamine formaldehyde resin.

Preferably, constituent (iv) is a polymer having a non-polar as well as a polar moiety. Preferably, these two moieties are sterically separated from each other within the backbone of the polymer. The polar moiety has a hydrophilic character, whereas the non-polar moiety has a hydrophobic character. Constituent (iv) has phase compatabilizing properties as it is able to function as phase mediator/phase promoter between hydrophilic constituents or parts/groups of constituents present in the coating material composition—via its hydrophilic moiety—and hydrophobic constituents or parts/groups of constituents present in the coating material composition—via its hydrophobic moiety. For example, polymer PMP can be at least one polymer selected from the group of polyurethanes, polyesters, (meth)acrylic (co)polymers and polymers composed of at least two of the structural units of these polymers, wherein each of these polymers used as polymer PMP preferably has at least one aforementioned polar moiety and at least one aforementioned non-polar moiety. Preferably, polymer PMP is obtainable by copolymerization of at least two different kinds of monomers in subsequent polymerization steps, each monomer having at least one ethylenically unsaturated carbon-carbon double bond, the first step involving a polymerization of at least one monomer being a non-polar monomer, for example a monomer having a low solubility (g/1) in water at 20° C. such as styrene, the second subsequent step involving a polymerization of at least one monomer being a polar monomer, for example a monomer having a high solubility (g/1) in water at 20° C. such as acrylic acid. The polymerization may be performed in presence of a polyurethane, which may or may not have, preferably does not have, ethylenically unsaturated carbon-carbon double bonds. Suitable polymers which can be used as constituents (iv) are, for example, known from DE 4437535 A1. In particular, constituent (iv) is a polyurethane poly(meth)acrylate.

Preferably, constituent (iv) is present in the first coating material composition F1 in an amount in a range of from 1.5 to 20 wt.-%, more preferably of from 2.5 to 18 wt.-%, even more preferably of from 3.5 to 15 wt.-%, in each case based on the total binder solids of the coating material composition. Preferably, the same applies to any of the further coating material compositions different from F1 and from one another provided in step (5) of the inventive method, and similarly in case of an at least one-fold repetition as defined in optional step (9).

### Constituent (v)

The first coating material composition F1 optionally comprises at least one additive A as constituent (v) such as at least one crosslinking catalyst CLC1, which is capable of catalyzing the crosslinking reaction of the crosslinkable functional groups of polymer P1 with the functional groups of crosslinking catalyst CA1. Likewise, each of the further coating material compositions provided in the inventive method may optionally comprise additive A and/or at least one additive being different from additive A. As outlined hereinbefore, if, e.g. crosslinking catalysts are used as additives (v) it is possible that these may exhibit further functions besides their catalytic activity. For example, in case blocked or unblocked sulfonic acids are used as crosslinking catalysts, these may also function as surfactants and thus also have an influence on the surface tension as a further example of a to be a measured property. This principle applies to all additives used as constituent (v).

The concept of an “additive” is known to the skilled person, from Römpp Lexikon “Lacke und Druckfarben”, Thieme Verlag, 1998, page 13, for example.

Examples of additives are reactive diluents, light stabilizers, crosslinking catalysts, antioxidants, deaerators, emulsifiers, surface-active agents such as surfactants, wetting agents and dispersants, and also thickeners, thixotropic agents, plasticizers, lubricity and antiblocking additives, slip additives, polymerization inhibitors, initiators for free-radical polymerizations, adhesion promoters, flow control agents, film-forming auxiliaries, sag control agents (SCAs), flame retardants, corrosion inhibitors, siccatives, thickeners, biocides and/or matting agents.

Preferably, if constituent (v) is present, it is a constituent that contributes to the total solids content of the respective coating material composition such as coating material composition F1, more preferably that contributes to the total binder solids content of the respective coating material composition such as coating material composition F1.

A preferred constituent (v) is at least one rheology additive and/or at least one crosslinking catalyst CLC1. The term “rheology additive” as well is known to the skilled person, from Römpp Lexikon “Lacke und Druckfarben”, Thieme Verlag, 1998, page 497, for example. The terms “rheology additive”, “rheological additive” and “rheology assistant” are interchangeable here. The additive optionally present as constituent (v) is preferably selected from the group consisting of flow control agents, surface-active agents such as surfactants, wetting agents and dispersants, and also thickeners, thixotropic agents, plasticizers, lubricity and antiblocking additives, and mixtures thereof. These terms are likewise known to the skilled person, from Römpp Lexikon, “Lacke und Druckfarben”, Thieme Verlag, 1998, for example. Flow control agents are components which by lowering the viscosity and/or surface tensions help coating composition materials to form films which flow out evenly. Wetting agents and dispersants are components which lower the surface tension or, generally, the interfacial tension. Lubricity and antiblocking additives are components which reduce mutual sticking (blocking).

Preferably, at least one sulfonic acid such as an unblocked sulfonic acid or a blocked sulfonic acid, more preferably a blocked sulfonic acid, is used as catalyst CLC1. Examples of unblocked sulfonic acids are para-toluenesulfonic acid (pTSA), methanesulfonic acid (MSA), dodecylbenzene sulfonic acid (DDBSA), dinonylnaphthalene disulfonic acid (DNNDSA), and mixtures thereof. Blocking can be performed by making use of ammonium salts and/or organic amines. Alternatively, blocking may also be performed by making use of epoxides that form β-OH-sulfonates when reversibly reacted with sulfonic acids.

As it is desired to use a rather non-complex standard coating material composition as composition F1, coating material composition F1 preferably does not contain any other constituents besides (i), (ii), (iii) and optionally (iv) and/or (v) and/or (vi). Preferably, the same applies to any of the further coating material compositions different from F1 and from one another provided in step (5) of the inventive method, and similarly in case of an at least one-fold repetition as defined in optional step (9).

The optionally present constituent (v) can be used in the known and customary proportions. Preferably, their content, based on the total weight of the coating material composition is 0.01 to 20.0 wt.-%, more preferably 0.05 to 15.0 wt.-%, particularly preferably 0.1 to 10.0% by weight, most preferably from 0.1 to 7.5% by weight, especially from 0.1 to 5.0% by weight and most preferably from 0.1 to 2.5% by weight.

### Constituent (vi)

The first coating material composition F1 optionally comprises at least one pigment P11 and/or at least one filler F11 as constituent (vi). Likewise, each of the further coating material compositions provided in the inventive method may optionally comprise pigment P11 and/or filler F11 and/or at least one pigment being different from pigment P11 and/or at least filler being different from filler F11. Preferably, however, the first coating material composition F1 does not comprise any pigments and/or fillers.

The term “pigment” is known to the skilled person, for example from DIN 55943 (date: October 2001). A “pigment” in the sense of the present invention refers preferably to a component in powder or flake form which is substantially, preferably entirely, insoluble in the medium surrounding them, such as in any of the coating material compositions. Pigments are preferably colorants and/or substances which can be used as pigment on account of their magnetic, electrical and/or electromagnetic properties. Pigments differ from “fillers” preferably in their refractive index, which for pigments is ≥1.7. The term “filler” is known to the skilled person, from DIN 55943 (date: October 2001), for example. For fillers their refractive index is <1.7.

The term “pigment” includes color pigments and effect pigments and color and effect pigments. Effect pigments are preferably pigments which have an optical effect or a color and optical effect. Examples of effect pigments are platelet-shaped metallic effect pigments such as platelet-shaped aluminum pigments, gold bronzes, fire-colored bronzes and/or iron oxide-aluminum pigments, perglaze pigments and/or metal oxide-mica pigments (mica). A person skilled in the art is familiar with the concept of color pigments. The terms “coloring pigment” and “color pigment” are interchangeable. As a color pigment inorganic and/or organic pigments can be used. Preferably, the color pigment is an organic color pigment. Particularly preferred color pigments used are white pigments, colored pigments and/or black pigments. Examples of suitable fillers are kaolin, dolomite, calcite, chalk, calcium sulfate, barium sulfate, talc, silicic acids, in particular pyrogenic silicic acids, hydroxides such as aluminum hydroxide or magnesium hydroxide or organic fillers such as textile fibers, cellulose fibers and/or polyolefine fibers; in addition, reference is made to Römpp Lexikon Lacke und Druckfarben, Georg Thieme Verlag, 1998, pages 250 ff., “Fillers”.

Preferably, the at least one optional filler F11 is present in the coating material composition F1 in an amount in the range of from 1.0 to 40 wt.-%, more preferably of from 2.0 to 35 wt.-%, still more preferably of from 5.0 to 30 wt.-%, in each case based on the total weight of the coating composition. Preferably, the at least one pigment P11 is present in the coating material composition F11 in an amount in the range of from 1.0 to 40 wt.-%, more preferably of from 2.0 to 35 wt.-%, still more preferably of from 5.0 to 30 wt.-%, in each case based on the total weight of the coating composition. The same preferably applies to any pigments and/or fillers being present in any of the further coating material compositions different from F1.

### Optional Step (2)

In optional step (2) the first coating material composition F1 is applied onto at least one optionally pre-coated surface of a substrate to form a wet coating film on said surface of the substrate. This step is performed in case a property of a wet coating film is intended to be measured in step (4).

The method of the invention is particularly suitable for the coating of automotive vehicle bodies or parts thereof including respective metallic substrates, but also plastic substrates. Consequently, the preferred substrates are automotive vehicle bodies or parts thereof.

Suitability as metallic substrates used in accordance with the invention are all substrates used customarily and known to the skilled person. The substrates used in accordance with the invention are preferably metallic substrates, more preferably selected from the group consisting of steel, preferably steel selected from the group consisting of bare steel, cold rolled steel (CRS), hot rolled steel, galvanized steel such as hot dip galvanized steel (HDG), alloy galvanized steel (such as, for example, Galvalume, Galvannealed or Galfan) and aluminized steel, aluminum and magnesium, and also Zn/Mg alloys and Zn/Ni alloys, or are alternatively preferably plastic substrates such as polyolefin substrates, e.g. Stamylan® products.

Particularly suitable substrates are parts of vehicle bodies or complete bodies of automobiles for production.

The metallic substrate used in accordance with the invention is preferably a substrate pretreated with at least one metal phosphate such as zinc phosphate. A pretreatment of this kind by means of phosphating, which takes place normally after the substrate has been cleaned and before the substrate is electrodeposition-coated, is in particular a pretreatment step that is customary in the automobile industry.

As outlined above the substrate used may be a pre-coated substrate, i.e. a substrate bearing at least one cured coating film. The substrate used in step (1) can be pre-coated with a cured electrodeposition coating layer and/or a cured primer or filler layer.

Applying according to step (2) can be performed by conventional means such as dipping, brushing, doctor blading or preferably spraying.

### Optional Step (3)

Optionally, the inventive method further comprises a step (3), which is carried out after optional step (2) and before step (4). This step is performed in case a property of a partially dried coating film is intended to be measured in step (4). In said step (3) the wet coating film obtained after step (2) is optionally partially dried to form a partially dried coating film on the surface of the substrate. Partial drying is preferably performed by flashing-off, preferably for a period of 1 to 30 minutes, more preferably for a period of 1.5 to 25 minutes, in particular for a period of 2 to 20 minutes, most preferably for a period of 3 to 15 minutes. Preferably, step (3) is performed at a temperature not exceeding 100° C., more preferably at a temperature in the range of from 18 to 80° C.

The term “flashing off” or “flash-off” in the sense of the present invention means a partial drying, wherein the solvents and/or water are evaporated from the coating film to some extent before curing is carried out. No intended and in particular no complete curing is performed by the flashing-off. Thus, neither the wet coating film obtained after step (2) nor the partially dried coating film obtained after optional step (3) represents a cured coating film.

### Step (4)

In step (4) at least one property of the coating material composition provided in step (1) and/or of the wet coating film—if step (2) is performed—and/or of the partially dried coating film—if step (3) is performed—is measured.

Preferably, the at least one property measured in step (4) and optionally measured after an at least one-fold repetition as defined in step (9) is at least one property selected from the group consisting of onset temperature, shrinkage, spread, surface tension, viscosity, in particular high shear viscosity and/or low shear viscosity, dispersion stability, in particular with respect to particle size distribution, expansion coefficient, and zeta potential.

Onset temperature and offset time are measured properties associated with the heat capacity and can be determined by the DMA IV Delta measurements described hereinafter in the “Methods” section. Improvements of these properties are e.g. in particular a lowering of the onset temperature and/or of the offset time in order to allow a subsequent curing at lowered temperatures.

Viscosity, in particular high shear viscosity and/or low shear viscosity, is a property associated with the rheology. Viscosity can be measured by using a rheometer such as Rheomat RM 180 from Mettler-Toledo at 23° C. and by supplying a sample to the measuring plate of the instrument and then subjecting the sample to shear stress of a shear rate of 1000/s for 5 minutes (high shear). The shear stress is then reduced to a shear rate of 1/s for 5 minutes (low shear) and the profile of the sol curve against time can be measured.

The expansion coefficient is a property associated with the linear thermal expansion and can be measured according to ISO 11359-2:1999-10.

Dispersion stability is a property associated with the particle size distribution within with respect to the shear rate and/or temperature and can be measured by a particle size analysis via laser diffraction according to ISO 13320:2020-01.

The zeta potential is a property associated with the electrical potential and can be measured according to ISO 13099-2:2012-06.

Shrinkage means the changes in volume that occur during partial drying of a wet or coating film or curing of a partially dried coating film. Shrinkage can be measured as disclosed in paragraphs [0055] to [0061] of EP 3 099 423 B1.

Surface tension is determined according to ISO 19403-2:2017-06. Determination of the surface tension may include determination of the contact angle and of the electrical dipole moment, in particular in association with the polarity and the surface energy of the wet or partially dried coating films. This property also has an influence on the wetting behavior of the wet or partially dried coating film. Thus, measurement of this property preferably includes measurement of said wetting behavior. Moreover, as it will be outlined hereinafter, surface tension correlates to the wettability of the wet or partially dried coating film.

Spread is determined according to ISO 19403-2:2017-06 and DIN EN ISO 19403-5:2020-04 and means the spread of the wet or partially dried coating films.

Preferably, the at least one property measured in step (4), and optionally measured after an at least one-fold repetition as defined in step (9), correlates to at least one characteristic of the coating material composition provided in step (1) and/or of the wet coating film if step (2) is performed and/or or of the partially dried coating film if step (3) is performed, preferably correlates to at least one of the characteristics selected from the group consisting of reactivity, reaction time, in particular crosslinking reaction time, (both determined according to DMA IV Delta measurements according to ISO 11357:2018), appearance (determined according to DIN EN ISO 13803:2015-02), color stability and flop, in particular after storage, (determined according to DIN EN ISO/CIE 11664-1:2020-03, DIN EN ISO/CIE 11664-2:2020-03, DIN EN ISO/CIE 11664-3:2020-03, DIN EN ISO/CIE 11664-4:2020-03 and DIN EN ISO 11664-5:2011-07), levelling (determined according to DIN EN ISO 13803:2015-02; haze test), wettability (determined according to ISO 19403-2:2017-06), adhesion (determined according to DIN EN ISO 4624:2016-08), and cohesion (determined according to DIN EN ISO 2409:2019-09; cross-cut test) as well as atomizability, storage stability and circulation line stability. Atomizability of a coating material composition, i.e. its sprayability, can be determined by visually investigating the occurrence of specks of a wet and/or partially dried coating film: A mist-coat of the coating material composition is first applied onto an optionally pre-coated substrate for 30 seconds from a distance of 1.5 meters. After a flash-off time of 2 minutes at room temperature, the same coating material composition is applied in the form of 1.5 cross-coats, after which the resulting wet coating film is flashed-off for one minute at room temperature. Then, the same coating material composition is applied again in the form of one additional cross-coat. The resulting wet coating film is then flashed-off for one minute at room temperature and then dried for 10 minutes in a circulating air oven at 70° C. The occurrence of specks is then visually investigated and grades of 0 to 6 are given, wherein “6” represents a very high number of specks (insufficient atomizability) and “0” means that no specks can be detected (excellent atomizability). Storage stability can be determined by measuring the viscosity of a coating material composition after different storage times over a period of up to 360 days. The viscosity is measured at a shear rate of 1000/s at 23° C. by using a rotary viscometer (Rheomat RM 180 instrument from Mettler-Toledo) at 23° C. within one hour after the preparation of the respective coating material composition, and also after different periods of storage time such as after 2, 4, 8, 20, 30, 50, 90, 120, 150, 180, 210, 240, 270, 300, 330 and 360 days. Apart from the respective measurements, the samples are stored over the entire period at 23° C. without influence of external shearing forces and are shaken for one minute prior to the viscosity measurement. Circulation line stability is determined by introducing 20 liters of a coating material composition into a circulation line system. The coating material composition is then pumped in circulation for a period of 77.1 minutes at a system operating pressure of 10 bar and at a temperature of 21±2° C. After this time, corresponding to an exposure of turnovers (1 turnover (TO)=1 circulation of material in the circulation line), 1.5 liters of the coating material composition are removed for coating purposes. This procedure is repeated up to a coating-material exposure of 2000 TO. Circulation line stability can be evaluated by measuring the color values such as lightness values of a coating film obtained from the removed coating material composition, for example by using a spectrophotometer such as an MA6811 spectrophotometer from X-Rite and/or by measuring the viscosity, in particular high shear viscosity and/or low shear viscosity according to the method described hereinbefore.

For example, the measured onset temperature correlates to the reactivity of the coating material composition, in particular to the reactivity of at least constituents (i) and (ii) and optionally (v) contained therein. This also applies when the coating material composition has been applied as a wet or partially dried film. For example, the measured offset time correlates with the reaction time, in particular crosslinking reaction time, in particular at a given temperature and/or temperature profile, of the coating material composition, in particular of at least constituents (i) and (ii) and optionally (v) contained therein. This also applies when the coating material composition has been applied as a wet or partially dried film. For example, shrinkage correlates to appearance, flop, and levelling. For example, surface tension correlates to wettability, cohesion and adhesion as well as appearance (in particular stability against overspray). For example, spread correlates to appearance (stability against overspray). For example, viscosity, in particular high shear viscosity and/or low shear viscosity, correlates with levelling, wettability, appearance and atomizability. For example, dispersion stability, in particular with respect to particle size distribution, correlates with color stability, storage stability and circulation line stability. For example, the expansion coefficient correlates with levelling and appearance. For example, the zeta potential correlates with storage stability and appearance.

### Step (5)

In step (5) at least one, preferably precisely one, further coating material composition being different from the first coating material composition F1 in precisely one or at most two parameters is provided. Said parameters are selected from the group consisting of (a) partial or full exchange of polymer P1 with a further polymer being different from polymer P1 and having crosslinkable functional groups being reactive at least towards the functional groups of crosslinking agent CA1, (b) partial or full exchange of crosslinking agent CA1 with a further crosslinking agent being different from crosslinking agent CA1 and having functional groups being reactive at least towards the crosslinkable functional groups of polymer P1, (c) lowering or increasing the amount of polymer P1 and/or of the polymer being different from polymer P1, which has been used for partial or full exchange of polymer P1 as defined in (a) in the further coating material composition, (d) lowering or increasing the amount of crosslinking agent CA1 and/or of the crosslinking agent being different from CA1, which has been used for partial or full exchange of CA1 as defined in (b) in the further coating material composition, (e) partial or full exchange of additive A if present such as of a crosslinking catalyst CLC1 with a further additive being different from additive A such as a crosslinking catalyst being different from crosslinking catalyst CLC1, and (f) lowering or increasing the amount of additive A if present such as of a crosslinking catalyst CLC1 and/or of the additive being different from additive A such as of a crosslinking catalyst being different from CLC1, which has been used for partial or full exchange of additive A such as of crosslinking catalyst CLC1 as defined in (e) in the further coating material composition, (g) partial or full exchange of pigment P11 and/or filler F11 if present with a further pigment and/or filler being different from pigment P11 and/or filler F11, and (h) lowering or increasing the amount of pigment P11 and/or filler F11 if present and/or of the pigment and/or filler being different from pigment P11 and/or filler F11 if present, which has been used for partial or full exchange of pigment P11 and/or filler F11 as defined in (g) in the further coating material composition. Preferably, as additives A and as any further additives different from additive A, crosslinking catalysts are used.

Preferably, the parameter(s) selected in step (5) and, optionally, during an at least one-fold repetition as defined in step (9), is/are associated with partial or full exchange and/or lowering or increasing of the amount of at least one constituent that contributes to the total solid content of the coating material composition, such as constituents (i) and (ii) and if present constituent (v) and/or (vi) in the coating material composition F1, preferably that contributes to the total binder solid content of the coating material composition, such as constituents (i) and (ii) and if present constituent (v) in the coating material composition F1.

If for example, polymer P1 present in coating material composition F1 is fully exchanged with another polymer being different from P1, this exchange is considered to be a one parameter change. However, in practice it is sometimes necessary to perform two modification steps: If, for example, polymer P1 has been used in a first water-based dispersion having a solid content of 50 wt.-% P1 and the other polymer being different from P1 is provided in a second water-based dispersion having a solids content of 75 wt.-% a less amount of the second dispersion needs to be used compared to the amount of the first dispersion that has been used for preparing F1. Thus, additional water has to be added so that—in total—both coating material compositions contain the same constituents in the same total amounts with the exception of the exchange of polymer P1 with the other polymer. However, in total only one parameter has been changed (namely the exchange of polymer P1 with another polymer).

Preferably, the at least one further coating material composition provided in step (5) and, optionally, provided after an at least one-fold repetition as defined in step (9), is different from the first coating material composition F1 in precisely one or at most two parameters, preferably in precisely one parameter, wherein said parameters are selected from the group consisting of (a) partial or full exchange of polymer P1 with a further polymer being different from polymer P1 and having crosslinkable functional groups being reactive at least towards the functional groups of crosslinking agent CA1, (b) partial or full exchange of crosslinking agent CA1 with a further crosslinking agent being different from crosslinking agent CA1 and having functional groups being reactive at least towards the crosslinkable functional groups of polymer P1, and (e) partial or full exchange of crosslinking catalyst CLC1 with a further crosslinking catalyst being different from crosslinking catalyst CLC1, as well as (f) lowering or increasing the amount of crosslinking catalyst CLC1 and/or of the crosslinking catalyst being different from CLC1, which has been used for partial or full exchange of CLC1 as defined in (e) in the further coating material composition.

Preferably,
the crosslinkable functional groups of polymer P1 being present as constituent (i) in the first coating material composition F1 and the crosslinkable functional groups of each polymer being different from polymer P1 and being present in any of the further coating material compositions provided in step (5) and, optionally, after an at least one-fold repetition as defined in step (9), are identical, preferably are in each case selected from hydroxyl and/or acid groups, more preferably represent in each case at least partially hydroxyl groups, and
the functional groups of crosslinking agent CA1 being present as constituent (ii) in the first coating material composition F1 and the functional groups of each crosslinking agent being different from crosslinking agent CA1 and being present in any of the further coating material compositions provided in step (5) and, optionally, after an at least one-fold repetition as defined in step (9), are identical, preferably are in each case selected from imino groups, alkylol groups, etherified alkylol groups and optionally blocked isocyanate groups, more preferably represent in each case at least partially etherified alkylol groups.

### Optional Step (6)

In optional step (6) the further coating material composition provided in step (5) is optionally applied onto at least one optionally pre-coated surface of a substrate to form a wet coating film on said surface of the substrate. This step is performed in case a property of a wet coating film is intended to be measured in step (8). The same substrates named hereinbefore can also be used in step (6) by the same conventional application means mentioned above.

Applying according to step (6) can be performed by conventional means such as dipping, brushing, doctor blading or preferably spraying.

### Optional Step (7)

Optionally, the inventive method further comprises a step (7), which is carried out after step (6) and before step (8). This step is performed in case a property of a partially dried coating film is intended to be measured in step (8). In said step (7) the wet coating film obtained after step (6) is optionally partially dried to form a partially dried coating film on the surface of the substrate. Partial drying is preferably performed by flashing-off, preferably for a period of 1 to 30 minutes, more preferably for a period of 1.5 to 25 minutes, in particular for a period of 2 to 20 minutes, most preferably for a period of 3 to 15 minutes. Preferably, step (7) is performed at a temperature not exceeding 100° C., more preferably at a temperature in the range of from 18 to 80° C.

As outlined hereinbefore the term “flashing off” in the sense of the present invention means a partial drying. No intended and in particular no complete curing is performed by the flashing-off. Thus, neither the wet coating film obtained after optional step (6) nor the partially dried coating film obtained after optional step (7) represents a cured coating film.

### Step (8)

In step (8) at least one property of the coating material composition provided in step (5) and/or of the wet coating film—if step (6) is performed—and/or of the partially dried coating film—if step (7) is performed—is measured. The at least one property is the same property as measured in step (4).

Preferably, the at least one property measured in step (8) and optionally measured after an at least one-fold repetition as defined in step (9) is at least one property selected from the group consisting of onset temperature, shrinkage, spread, surface tension, viscosity, in particular high shear viscosity and/or low shear viscosity, dispersion stability, in particular with respect to particle size distribution, expansion coefficient, and zeta potential.

These properties have been already defined and/or described hereinbefore and methods for determination of these properties have also been named.

Preferably, the at least one property measured in step (8), and optionally measured after an at least one-fold repetition as defined in step (9), correlates to at least one characteristic of the coating material composition provided in step (5) and/or of the wet coating film if step (6) is performed and/or or of the partially dried coating film if step (7) is performed, preferably correlates to at least one of the characteristics selected from the group consisting of reactivity, reaction time, in particular crosslinking reaction time, (both determined according to DMA IV Delta measurements according to ISO 11357:2018), appearance (determined according to DIN EN ISO 13803:2015-02), color stability and flop, in particular after storage, (determined according to DIN EN ISO/CIE 11664-1:2020-03, DIN EN ISO/CIE 11664-2:2020-03, DIN EN ISO/CIE 11664-3:2020-03, DIN EN ISO/CIE 11664-4:2020-03 and DIN EN ISO 11664-5:2011-07), levelling (determined according to DIN EN ISO 13803:2015-02; haze test), wettability (determined according to ISO 19403-2:2017-06), adhesion (determined according to DIN EN ISO 4624:2016-08), and cohesion (determined according to DIN EN ISO 2409:2019-09; cross-cut test) as well as atomizability, storage stability and ring circulation line stability. Methods for determining atomizability, storage stability and circulation line stability have already been mentioned hereinbefore.

For example, the measured onset temperature correlates to the reactivity of the coating material composition, in particular to the reactivity of at least constituents (i) and (ii) and optionally (v) contained therein. This also applies when the coating material composition has been applied as a wet or partially dried film. For example, the measured offset time correlates with the reaction time, in particular crosslinking reaction time, in particular at a given temperature and/or temperature profile, of the coating material composition, in particular of at least constituents (i) and (ii) and optionally (v) contained therein. This also applies when the coating material composition has been applied as a wet or partially dried film. For example, shrinkage correlates to appearance, flop, and levelling. For example, surface tension correlates to wettability, cohesion and adhesion as well as appearance (in particular stability against overspray). For example, spread correlates to appearance (stability against overspray). For example, viscosity, in particular high shear viscosity and/or low shear viscosity, correlates with levelling, wettability, appearance and atomizability. For example, dispersion stability, in particular with respect to particle size distribution, correlates with color stability, storage stability and circulation line stability. For example, the expansion coefficient correlates with levelling and appearance. For example, the zeta potential correlates with storage stability and appearance.

### Optional Step (9)

In optional step (9) steps (5) to (8) are repeated optionally at least once, wherein each of the further coating compositions provided in this manner is different from one another and different from coating material composition F1. The number of repetitions is not limited and can be, for example, in a range of from 1 to 1000 or of from 1 to 100 repetitions.

### Step (10)

In step (10) the results of the properties measured in steps (4) and (8) and optionally of the results of the properties measured in case of an at least one-fold repetition as defined in step (9) are incorporated into a preferably electronic database. Preferably, step (10) is performed with support of at least one software. The database can be updated constantly while performing the inventive method.

### Step (11)

In step (11) at least one of the measured properties present in the preferably electronic database due to incorporation step (10) is selected.

Preferably, the at least one property selected in step (11) is at least one property selected from the group consisting of onset temperature and offset time, shrinkage, spread, surface tension, viscosity, in particular high shear viscosity and/or low shear viscosity, dispersion stability, in particular with respect to particle size distribution, expansion coefficient, and zeta potential.

These properties have been already defined and/or described hereinbefore and methods for determination of these properties have also been named.

Preferably, the at least one property selected in step (11) correlates to at least one characteristic of a coating material composition and/or of a wet coating film and/or or of a partially dried coating film, preferably correlates to at least one of the characteristics selected from the group consisting of reactivity, reaction time, in particular crosslinking reaction time (both determined according to DMA IV Delta measurements according to ISO 11357:2018), appearance (determined according to DIN EN ISO 13803:2015-02), color stability and flop, in particular after storage, (determined according to DIN EN ISO/CIE 11664-1:2020-03, DIN EN ISO/CIE 11664-2:2020-03, DIN EN ISO/CIE 11664-3:2020-03, DIN EN ISO/CIE 11664-4:2020-03 and DIN EN ISO 11664-5:2011-07), levelling (determined according to DIN EN ISO 13803:2015-02; haze test), wettability (determined according to ISO 19403-2:2017-06), adhesion (determined according to DIN EN ISO 4624:2016-08), and cohesion (determined according to DIN EN ISO 2409:2019-09; cross-cut test) as well as atomizability, storage stability and circulation line stability. Methods for determining atomizability, storage stability and circulation line stability have already been mentioned hereinbefore.

For example, the measured onset temperature correlates to the reactivity of the coating material composition, in particular to the reactivity of at least constituents (i) and (ii) and optionally (v) contained therein. This also applies when the coating material composition has been applied as a wet or partially dried film. For example, the measured offset time correlates with the reaction time, in particular crosslinking reaction time, in particular at a given temperature and/or temperature profile, of the coating material composition, in particular of at least constituents (i) and (ii) and optionally (v) contained therein. This also applies when the coating material composition has been applied as a wet or partially dried film. For example, shrinkage correlates to appearance, flop, and levelling. For example, surface tension correlates to wettability, cohesion and adhesion as well as appearance (in particular stability against overspray). For example, spread correlates to appearance (stability against overspray). For example, viscosity, in particular high shear viscosity and/or low shear viscosity, correlates with levelling, wettability, appearance and atomizability. For example, dispersion stability, in particular with respect to particle size distribution, correlates with color stability, storage stability and circulation line stability. For example, the expansion coefficient correlates with levelling and appearance. For example, the zeta potential correlates with storage stability and appearance.

### Step (12)

In step (12) the results of the at least one selected property measured according to steps (4) and (8) and optionally after an at least one-fold repetition as defined in step (9) are evaluated and compared with each other. Preferably, this is performed by means of at least one software.

Preferably, evaluation and comparison step (12) as well as step (13) make use of all results of the at least one property selected in step (11), which are present in the electronic database.

### Step (13)

In step (13) the information obtained from evaluation and comparison step (12) are used for formulating and providing at least one new coating formulation being different from the first coating material composition F1 and from each of the further coating material compositions obtained after step (5) and from any additional coating material composition obtained optionally after an at least one-fold repetition as defined in step (9), wherein the at least one new coating formulation as such and/or a wet or partially dried coating film obtained therefrom shows an improvement of the at least one property selected in step (11), when measured as defined in step (4) and/or (8).

Preferably, the at least one new coating formulation provided in step (13) is different from the first coating material composition F1 and from any of the further coating material compositions obtained after step (5) and from any coating material composition obtained optionally after an at least one-fold repetition as defined in step (9), in at least one of the parameters as defined within step (5).

Preferably, the at least one new coating formulation provided in step (13) as such and/or a wet or partially dried coating film obtained therefrom not only shows an improvement of the at least one property selected in step (11), preferably of at least one property as defined hereinbefore when measured as defined in step (4) and/or (8), but also shows an improvement of at least one characteristic of the coating formulation as such and/or of a wet or partially dried coating film obtained therefrom, said characteristic preferably being a characteristic as defined hereinbefore, and being in correlation to said at least one property.

### Inventive Uses

A further subject-matter of the present invention is a use of the inventive method for developing and providing new coating formulations in the automotive field, said new coating formulations preferably being basecoat compositions, in particular aqueous basecoat compositions.

All preferred embodiments described hereinbefore in connection with the inventive method are also preferred embodiments with regard to the aforementioned inventive use.

A further subject-matter of the present invention is a use of the inventive method for investigating the influence of film-forming polymers (i), said polymers having crosslinkable functional groups, and/or of crosslinking agents (ii) having functional groups, which are reactive at least towards the crosslinkable functional groups of the polymers, and/or of additives (v) such as crosslinking catalysts catalyzing a crosslinking reaction between (i) and (ii) and/or of pigments and/or fillers (vi) on properties of coating material compositions as such and/or of wet or partially dried coating films obtained therefrom, said coating material compositions containing at least constituents (i) and (ii) and optionally (v) and/or (vi). The inventive use preferably aims at improvement of these properties.

All preferred embodiments described hereinbefore in connection with the inventive method as well as the above defined inventive use are also preferred embodiments with regard to the inventive use of the inventive method for investigating the aforementioned influence.

### Methods

### 1. Determining the Non-Volatile Fraction

The amount of solid content (non-volatile matter, solid fraction) including the total solid content is determined via DIN EN ISO 3251:2019-09 at 110° C. for 60 min.

### 2. DMA IV Delta Measurements

DMA (dynamic mechanical analysis) IV Delta measurements of wet coating films obtained from coating compositions were used in order to determine the onset temperature (E′ progress, [° C.], corresponding to reactivity) and the offset time (extrapolated, [min], corresponding to reaction time) of the wet films. The onset temperature measurements were performed at a frequency of 1 Hz (amplitude: 0.2%) and in a range of from 25° C. to 200° C. (2° C./min). The offset time measurements were performed at a frequency of 1 Hz (amplitude: 0.2%) and in a range of from 25° C. to 120° C. (24° C./min) as well as in a range of from 25° C. to 120° C. (6° C./min) and for 63 min (isothermally at 140° C.). The following parameters were determined: onset temperature (extrapolated onset temperature of the network structure from E′-progression and extrapolated onset temperature of the network structure from tan δ progression), offset time (extrapolated offset time [min] of the network structure from E′ progression in linear application, ratio of storage moduli after 70 minutes and after 20 minutes. The measurements were performed according to ISO 11357:2018.

### EXAMPLES

The following examples further illustrate the invention, but are not to be construed as limiting its scope. ‘Pbw’ means parts by weight. If not defined otherwise, ‘parts’ means ‘parts by weight’.

### 1. Preparation of Coating Compositions

1.1 A number of coating compositions (I1, I2 and I3) have been prepared. The constituents listed in Table 1a have been mixed under stirring in a dissolver in the sequence given in said Table to prepare the coating compositions.

TABLE 1a
Coating compositions I1 to I3

| | | | | Amount binder solids | | |
|---|---|---|---|---|---|---|
| | | | | with respect to total | | |
| | Amount [pbw] | | | binder solids [wt.-%] | | |
| Constituent | I1 | I2 | I3 | I1 | I2 | I3 |
| Binder 1 | 7.41 | | | 50 | | |
| Binder 2 | | 5.55 | | | 50 | |
| Binder 3 | | | 4.44 | | | 50 |
| Crosslinker | 8.03 | 8.03 | 8.03 | 36 | 36 | 36 |
| Additional binder | 5.00 | 5.00 | 5.00 | 14 | 14 | 14 |
| Butyl glycol | 0.77 | 1.14 | 2.25 | | | |

Binder 1 is an aqueous polyester dispersion prepared as disclosed in example D of DE 4009858 A1 except that butyl glycol has been used instead of butanol and having a solid content of 60 wt.-%. Binder 2 is a polyether dispersion prepared as disclosed in example ER1 of WO 2017/097642 A1 and having a solid content of 80 wt.-%. Binder 3 is a polyether dispersion prepared as disclosed in example ER1 of WO 2017/121683 A1 and having a solid content of 99.9 wt.-%. Luwipal® 052 is a commercially available melamine formaldehyde resin crosslinker (BASF SE) and has a solid content of 77 wt.-%. A polyurethane poly(meth)acrylate dispersion has been used as additional binder. This dispersion was prepared as disclosed in example D of DE 4437535 A1 and has a solid content of 40 wt.-%. Butyl glycol was merely added to ensure that each of 11 to 13 has the same binder solids content and the same solvent composition as the binders 1 to 3 used for preparing 11 to 13 each have different solids contents and contain different amounts of butyl glycol.
1.2 A number of further coating compositions (I4, I5 and I6) have been prepared. The constituents listed in Table 1b have been mixed under stirring in a dissolver in the sequence given in said Table to prepare the coating compositions.

TABLE 1b
Coating compositions I4 to I6

| | | | | Amount binder solids | | |
|---|---|---|---|---|---|---|
| | | | | with respect to total | | |
| | Amount [pbw] | | | binder solids [wt.-%] | | |
| Constituent | I4 | I5 | I6 | I4 | I5 | I6 |
| Binder 1 | 7.41 | 7.41 | 7.41 | 50 | 50 | 50 |
| Crosslinker | 8.03 | 8.03 | 8.03 | 36 | 36 | 36 |
| Additional binder | 5.00 | 5.00 | 5.00 | 14 | 14 | 14 |
| Butyl glycol | 0.77 | 1.14 | 2.25 | | | |
| Catalyst 1 | — | 0.77 | — | | | |
| Catalyst 2 | — | — | 1.33 | | | |
| Iso-propanol | 0.23 | — | 0.23 | | | |
| n-propanol | 0.10 | — | 0.10 | | | |
| Deionized water | 1.15 | 1.08 | — | | | |

Binder 1, crosslinker and additional binder have already been identified hereinbefore in item 1.1. Catalyst 1 is a mixture of 30.3 wt.-% iso-propanol, 10 wt.-% deionized water, 13.6 wt.-% n-propanol, 30.3 wt.-% para-toluene sulfonic acid and 15.8 wt.-% of a solution of 2-amino-2-methyl-1-propanol in deionized water (90 wt. %). Catalyst 2 is a mixture of 17.2 pbw para-toluene sulfonic acid, 71.47 pbw water and 10.95 pbw of a solution of ammonia in deionized water (15 wt.-%). Iso-propanol, n-propanol and/or deionized water were added to ensure that each of 14 to 16 has the same solvent composition.

### 2. Investigation of Properties of the Coating Compositions

2.1 Influence of binders on reactivity and reaction time

The influence of the binders used for preparation of coating compositions I1 to I3 on onset temperature and offset time has been investigated by DMA IV Delta measurements according to the method identified hereinbefore in the ‘Methods’ section. The results are summarized in Table 2a:

TABLE 2a
DMA IV Delta results

| | Onset temperature (E′ | Offset time, extrapolated |
|---|---|---|
| Coating | progress) [° C.] | [min] |
| composition | Reactivity | Reaction time |
| I1 | 94 | 25 |
| I2 | 96 | 16 |
| I3 | 90 | 10 |

These results show that differences in reactivity (onset temperatures) and reaction time such as crosslinking time (offset time) resulting from the different coating compositions I1 to I3 are obtained. The differences in the measured parameters can be directly related to the different polymers used as binders in I1 to I3 as the coating compositions otherwise contain the same constituents.

These results can be used for the tailor-made provision of new coating material formulations, which are much more complex than the systems I1 to I3.

For example, both binder 1 and binder 2 can be used for preparing aqueous basecoat compositions as it is, e.g., disclosed in WO 2017/097642 A1 (basecoat example 1 containing polyester binder 1 vs. basecoat example E1 containing polyether binder 2 disclosed therein). It has been in particular found that an exchange of binder 1 with binder 2 leads to a lowering of the offset time as an improvement. This improvement of the offset time as a property of the coating material composition can be correlated to a shorter reaction time as measured by DMA IV Delta measurements for I2 vs. I1.

Similar observations apply in case of binder 1 vs. binder 3. Both binder 1 and binder 3 can be used for preparing aqueous basecoat compositions as it is, e.g., disclosed in WO 2017/121683 A1 (basecoat example V1 containing polyester binder 1 vs. basecoat example E1 containing polyether binder 3 disclosed therein). It has been found that an exchange of binder 1 with binder 3 leads to a lowering of both the onset temperature and the offset time as an improvement. This improvement of the offset time as a property of the coating material composition and of the onset temperature as a property of the coating material composition can be correlated to the shorter reaction time and to a higher reactivity as measured by DMA IV Delta measurements for I3 vs. I1.
2.2 In addition to investigating the influence of the aforementioned constituents on the reactivity and reaction time, further effects of the constituents can also be determined. For this purpose coating compositions I1, I2 and I3 were each adjusted to the same high shear viscosity (100 mPas at a shear rate of 1000 s⁻¹). The required amount of deionized water and the resulting solid content is shown in the following Table 2b.

TABLE 2b

| | Amount of deionized water | |
|---|---|---|
| | [pbw] | Solid content [wt.-%] |
| I1 | 13.9 | 34.6 |
| I2 | 15.1 | 33.5 |
| I3 | 19.6 | 29.8 |

The results obtained above can also be used directly to further optimize the properties.

For example, as illustrated by Table 2b these properties can be used to shift the solids content of a coating formulation in one direction or the other, depending on the effect desired and required.
2.3 Influence of catalysts on reactivity and reaction time

The influence of catalysts for preparation of coating compositions I4 to I6 has been investigated by DMA IV Delta measurements according to the method identified hereinbefore in the ‘Methods’ section. The results are summarized in Table 2c:

TABLE 2c
DMA IV Delta results

| | Onset temperature (E′ | Offset time, extrapolated |
|---|---|---|
| | progress) [° C.] | [min] |
| Corresponds to | Reactivity | Reaction time |
| I4 | 97 | 10 |
| I5 | 95 | 11 |
| I6 | 91 | 9 |

These results show that the presence or non-presence of a catalyst can be detected due to differences in reactivity (lower onset temperatures) and reaction time (shorter crosslinking time) for the different coating compositions I4 to I6.

These results can be used for the tailor-made provision of new coating material formulations, which are much more complex than the systems I4 to I6. For example, these results can be used for providing new coating material formulations that can be used as basecoats and that allow a curing to take place at low temperatures, in particular at lower temperatures compared to the ones required when I4 is used, and/or that allow shorter times at elevated temperature, in particular at curing temperatures as low as possible and/or at shorter times at elevated temperatures, as disclosed e.g. in WO 2018/036855 A1.

1. A method of screening coating material compositions for development of new coating formulations in the automotive field, said method comprising:
(1) providing a first coating material composition F1, the first coating material composition F1 comprising
(i) at least one film-forming polymer P1, said polymer P1 having crosslinkable functional groups,
(ii) at least one crosslinking agent CA1 having functional groups, which are reactive at least towards the crosslinkable functional groups of polymer P1,
(iii) water and/or at least one organic solvent,
(iv) optionally at least one phase compatibilizing polymer PMP,
(v) optionally at least one additive A, and
(vi) optionally at least one pigment PI1 and/or at least one filler FI1,
wherein the constituents (i), (ii), (iii), (iv) and (v) as well as (vi) are each different from one another,
(2) optionally applying the first coating material composition F1 onto at least one optionally pre-coated surface of a substrate to form a wet coating film on said surface of the substrate,
(3) optionally drying the wet coating film obtained after step (2) to form a partially dried coating film on the surface of the substrate,
(4) measuring at least one property of the coating material composition provided in step (1) and/or of the wet coating film if step (2) is performed and/or of the partially dried coating film if step (3) is performed,
(5) providing at least one further coating material composition being different from the first coating material composition F1 in precisely one or at most two parameters,
wherein said parameters are selected from the group consisting of (a) partial or full exchange of polymer P1 with a further polymer being different from polymer P1 and having crosslinkable functional groups being reactive at least towards the functional groups of crosslinking agent CA1, (b) partial or full exchange of crosslinking agent CA1 with a further crosslinking agent being different from crosslinking agent CA1 and having functional groups being reactive at least towards the crosslinkable functional groups of polymer P1, (c) lowering or increasing the amount of polymer P1 and/or of the polymer being different from polymer P1, which has been used for partial or full exchange of polymer P1 as defined in (a) in the further coating material composition, (d) lowering or increasing the amount of crosslinking agent CA1 and/or of the crosslinking agent being different from CA1, which has been used for partial or full exchange of CA1 as defined in (b) in the further coating material composition, (e) partial or full exchange of additive A if present with a further additive being different from additive A, (f) lowering or increasing the amount of additive A if present and/or of the additive being different from additive A if present, which has been used for partial or full exchange of additive A as defined in (e) in the further coating material composition, (g) partial or full exchange of pigment PI1 and/or filler FI1 if present with a further pigment and/or filler being different from pigment PI1 and/or filler FI1, and (h) lowering or increasing the amount of pigment PI1 and/or filler FI1 if present and/or of the pigment and/or filler being different from pigment PI1 and/or filler FI1 if present, which has been used for partial or full exchange of pigment PI1 and/or filler FI1 as defined in (g) in the further coating material composition,
(6) optionally applying the further coating material composition onto at least one optionally pre-coated surface of a substrate to form a wet coating film on said surface of the substrate,
(7) optionally drying the wet coating film obtained after step (6) to form a partially dried coating film on the surface of the substrate,
(8) measuring at least one property of the coating material composition provided in step (5) and/or of the wet coating film if step (6) is performed and/or or of the partially dried coating film if step (7) is performed, said at least one property being the same property as measured in step (4),
(9) optionally repeating steps (5) and (8) and optionally step (6) and/or step (7) at least once, wherein each of the further coating compositions provided is different from one another and different from coating material composition F1,
(10) incorporating the results of the properties measured in steps (4) and (8) and optionally of the results of the properties measured in case of an at least one-fold repetition as defined in step (9) into a database,
(11) selecting at least one of the measured properties present in the database due to incorporation step (10),
(12) evaluating and comparing the results of the at least one selected property measured according to steps (4) and (8) and optionally after an at least one-fold repetition as defined in step (9) with each other, and
(13) using the information obtained from evaluation and comparison step (12) for formulating and providing at least one new coating formulation being different from the first coating material composition F1 and from each of the further coating material compositions obtained after step (5) and from any coating material composition obtained optionally after an at least one-fold repetition as defined in step (9), wherein the at least one new coating formulation as such and/or a wet or partially dried coating film obtained therefrom shows an improvement of the at least one property selected in step (11), when measured as defined in step (4) and/or (8).

2. The method according to claim 1, characterized in that
the crosslinkable functional groups of polymer P1 being present as constituent (i) in the first coating material composition F1 and the crosslinkable functional groups of each polymer being different from polymer P1 and being present in any of the further coating material compositions provided in step (5) and, optionally, after an at least one-fold repetition as defined in step (9), are identical, and
the functional groups of crosslinking agent CA1 being present as constituent (ii) in the first coating material composition F1 and the functional groups of each crosslinking agent being different from crosslinking agent CA1 and being present in any of the further coating material compositions provided in step (5) and, optionally, after an at least one-fold repetition as defined in step (9), are identical.

3. The method according to claim 1, characterized in that the first coating material composition F1 and any of the further coating material compositions provided in step (5) and, optionally, obtained after an at least one-fold repetition as defined in step (9), are one-component (1K) or two-component (2K) coating material compositions.

4. The method according to claim 1, characterized in that the at least one crosslinking agent CA1 present as constituent (ii) in the first coating material composition F1 and any further at least one crosslinking agent being present in any of the further coating material compositions provided in step (5) and, optionally, obtained after an at least one-fold repetition as defined in step (9), are melamine aldehyde resins.

5. The method according to claim 1, characterized in that the at least one polymer P1 present as constituent (i) in the first coating material composition F1 and any further at least one film-forming polymer being present in any of the further coating material compositions provided in step (5) and, optionally, obtained after an at least one-fold repetition as defined in step (9), is selected from the group consisting of physically drying polymers, chemically crosslinking polymers, radiation curing polymers, and mixtures thereof.

6. The method according to claim 1, characterized in that the at least one polymer P1 present as constituent (i) in the first coating material composition F1 and any further at least one film-forming polymer being present in any of the further coating material compositions provided in step (5) and, optionally, obtained after an at least one-fold repetition as defined in step (9), are selected from the groups consisting of polyesters, poly(meth)acrylates, polyurethanes, polyureas, polyamides and polyethers.

7. The method according to claim 1, characterized in that the constituents (i) to (iii) and optionally (iv) and/or (v) and/or (vi) are the only constituents of the first coating material composition F1.

8. The method according to claim 1, characterized in that constituent (iv) is present in the first coating material composition F1.

9. The method according to claim 1, characterized in that the parameter(s) selected in step (5) and, optionally, during an at least one-fold repetition as defined in step (9), is/are associated with partial or full exchange and/or lowering or increasing of the amount of at least one constituent that contributes to the total solid content of each of the coating material compositions.

10. The method according to claim 1, characterized in that the at least one property measured in steps (4) and (8), and optionally measured after an at least one-fold repetition as defined in step (9), and selected in step (11), is at least one property selected from the group consisting of onset temperature, offset time, shrinkage, spread, surface tension, viscosity, high shear viscosity, low shear viscosity, and dispersion stability.

11. The method according to claim 1, characterized in that the at least one property measured in steps (4) and (8), and optionally measured after an at least one-fold repetition as defined in step (9), and selected in step (11), correlates to at least one characteristic of the coating material composition and/or of a wet or partially dried coating film obtained therefrom.

12. The method according to claim 1, characterized in that evaluation and comparison step (12) as well as step (13) make use of all results of the at least one property selected in step (11), which are present in the database.

13. The method according to claim 1, characterized in that the at least one new coating formulation provided in step (13) is different from the first coating material composition F1 and from any of the further coating material compositions obtained after step (5) and from any coating material composition obtained optionally after an at least one-fold repetition as defined in step (9), in at least one of the parameters as defined within step (5).

14. The method according to claim 1, characterized in that the at least one new coating formulation provided in step (13) as such and/or a wet or partially dried coating film obtained therefrom not only shows an improvement of the at least one property selected in step (11), but also shows an improvement of at least one characteristic of the coating formulation as such and/or of a wet or partially dried coating film obtained therefrom.

15. A method for investigating the influence of film forming polymers (i), the method comprising using the method of claim 1 to investigate the influence of film forming polymers (i), wherein said polymers have crosslinkable functional groups and/or crosslinking agents (ii) have functional groups, which are reactive at least towards the crosslinkable functional groups of the polymers, and/or of additives (v), and/or of pigments and/or fillers (vi) on properties of coating material compositions as such and/or of wet or partially dried coating films obtained therefrom, said coating material compositions containing at least constituents (i) and (ii) and optionally (v) and/or (vi).

16. The method according to claim 1, characterized in that
the crosslinkable functional groups of polymer P1 being present as constituent (i) in the first coating material composition F1 and the crosslinkable functional groups of each polymer being different from polymer P1 and being present in any of the further coating material compositions provided in step (5) and, optionally, after an at least one-fold repetition as defined in step (9), are identical and in each case selected from the group consisting of hydroxyl and/or acid groups, and
the functional groups of crosslinking agent CA1 being present as constituent (ii) in the first coating material composition F1 and the functional groups of each crosslinking agent being different from crosslinking agent CA1 and being present in any of the further coating material compositions provided in step (5) and, optionally, after an at least one-fold repetition as defined in step (9), are identical, and in each case selected from the group consisting of imino groups, alkylol groups, etherified alkylol groups and optionally blocked isocyanate groups.

17. The method according to claim 1, characterized in that the first coating material composition F1 and any of the further coating material compositions provided in step (5) and, optionally, obtained after an at least one-fold repetition as defined in step (9), are one-component (1K) coating material compositions for use as basecoat material compositions.

18. The method according to claim 1, characterized in that the at least one crosslinking agent CA1 present as constituent (ii) in the first coating material composition F1 and any further at least one crosslinking agent being present in any of the further coating material compositions provided in step (5) and, optionally, obtained after an at least one-fold repetition as defined in step (9), are melamine formaldehyde resins.

19. The method according to claim 1, characterized in that the at least one polymer P1 present as constituent (i) in the first coating material composition F1 and any further at least one film-forming polymer being present in any of the further coating material compositions provided in step (5) and, optionally, obtained after an at least one-fold repetition as defined in step (9), is selected from the group consisting of chemically non-self-crosslinking polymers.

20. The method according to claim 1, characterized in that constituent (iv) is present in the first coating material composition F1 when constituent (iii) represents at least partially water.
